SEQUENCE LISTING (4 sequences)

SEQ ID NO: 1 - DNA, 20 bp, Artificial Sequence
  Alb forward
tgcttttccaggggtgtgtt

SEQ ID NO: 2 - DNA, 23 bp, Artificial Sequence
  Alb reverse
ttacttcctgcactaatttggca

SEQ ID NO: 3 - DNA, 19 bp, Artificial Sequence
  Gapdh forward
gttgtctcctgcgacttca

SEQ ID NO: 4 - DNA, 19 bp, Artificial Sequence
  Gapdh reverse
ggtggtccagggtttctta

(19) United States
(12) Patent Application Publication
(10) Pub. No.: US 2023/0265166 A1
(43) Pub. Date: Aug. 24, 2023

(54) YOUNG AND UNDAMAGED HUMAN SERUM ALBUMIN IMPROVES LONGEVITY OF HUMAN

(71) Applicant: SHENZHEN PROTGEN LTD., Guangong (CN)

(72) Inventors: Yongzhang Luo, Beijing (CN); Yan FU, Beijing (CN); Jiaze TANG, Beijing (CN); Anji JU, Beijing (CN); Boya LI, Beijing (CN); Yuanchao GONG, Beijing (CN); Shaosen ZHANG, Beijing (CN); Guodong CHANG, Beijing (CN); Hui LI, Beijing (CN)

(21) Appl. No.: 18/041,319

(22) PCT Filed: Aug. 11, 2021
(86) PCT No.: PCT/CN2021/112093

§ 371 Date: Feb. 10, 2023

(30) Foreign Application Priority Data
Aug. 11, 2020 (WO) PCT/CN2020/108496

(51) Int. Cl. C07K 14/765; A61K 38/38; A61P 21/00

(52) U.S. Cl. CPC C07K 14/765; A61K 38/385; A61P 21/00

(57) ABSTRACT

Provided is a method of extending lifespan and/or anti-aging in a subject, comprising the step of administering to the subject an effective amount of a preparation of young and undamaged human serum albumin (HSA), wherein the preparation exhibits at least one, preferably all, of the following properties: (1) higher ratio of free thiol in Cys-34 residue, (2) lower level of advanced glycation end-product (AGE), (3) lower level of carbonylation, and (4) lower level of homocysteinylation, as compared to an endogenous HSA preparation obtained from said subject.

### FIELD OF THE INVENTION

The present inventions relate to the field of biomedical sciences, in particular, to the use of young and undamaged human serum albumin (HSA) in improving longevity of human.

### BACKGROUND OF THE INVENTION

Longevity is an eternal pursuit of human beings. Tales of passionate seeking for immortality ran through the whole human history. Ironically, elixir was never found while aging continues. Understanding the mechanism of aging is essential for realizing the dream of longevity, but the complexity and systemic nature of aging make this process poorly understood.

### SUMMARY OF THE INVENTION

The present invention is partly based on the surprising finding that administration of young and undamaged recombinant mouse serum albumin (rMSA) in natural aging mouse model remarkably obtained significantly extended lifespan with increased skeletal muscle strength and cognitive ability compared with saline-treated groups.

In one aspect, the present invention provides a preparation of young and undamaged human serum albumin (HSA), wherein it exhibits at least one, preferably all, of the following properties: (1) higher ratio of free thiol in Cys-34 residue, (2) lower level of advanced glycation end-product (AGE), (3) lower level of carbonylation, and (4) lower level of homocysteinylation, as compared to an endogenous HSA preparation obtained from a young individual of human.

In another aspect, the present invention provides a method of extending lifespan and/or anti-aging in a subject, comprising the step of administering to the subject an effective amount of a preparation of young and undamaged human serum albumin (HSA), wherein the preparation exhibits at least one, preferably all, of the following properties: (1) higher ratio of free thiol in Cys-34 residue, (2) lower level of advanced glycation end-product (AGE), (3) lower level of carbonylation, and (4) lower level of homocysteinylation, as compared to an endogenous HSA preparation obtained from said subject.

### BRIEF DESCRIPTION OF THE DRAWINGS

FIG. 1. rMSA treatment increased the longevity in mice

(A and B) Survival curves for female (A) and male (B) mice in the 6 M group treated with rMSA or saline. (C) Representative images of mice treated with rMSA or saline.

(D and E) Survival curves of female (D) and male (E) mice in the 3 M group treated with rMSA or saline. (F and G) Survival curves of female (F) and male (G) mice in the 18 M group treated with rMSA or saline. Mice in the 6 M, 3 M, and 18 M groups (A-G) were treated with 20 mg rMSA per mouse or isometric saline every 3 weeks. (H and I) Survival curves of female (H) and male (I) mice in the 12 M group treated with 1.5 mg rMSA per gram of body weight or isometric saline every 3 weeks. Median survivals (in months, M) and percentage increases are indicated. p-value was calculated by the log-rank (Mantel-Cox) test. n, number of mice used for each analysis.

FIG. 2. The effects of rMSA on the function of skeletal muscle in mice

(A and B) The grip strength of female (A) and male (B) mice treated with body weight-adjusted dosage of rMSA or saline in the 12 M group. (C) The toluidine blue staining of gastrocnemius muscle. Scar bar, 50 μm. (D-G) The statistical results of data in (C). Saline-treated mice; rSMA-treated mice, female, n=4; male, n=6. Mean size of myofibers=cross-sectional area/the number of fibers. (H) The immunofluorescence staining for MHC1 (green) and 4′,6-diamidino-2-phenylindole (DAPI) (blue) in mice. Scale bar, 50 μm. (I and J) The intensity of fluorescence on MHC1 in (H) by using Image J. Mice were treated by rMSA 1.5 mg per gram of body weight or isometric saline every 3 weeks for 8 months. All graphs represent mean with SEM, with p values calculated by the two-tail t test. n, number of mice used for each analysis.

FIG. 3. Effects of rMSA on the cognitive ability of mice

(A and C) Measurements of the primary escape rate (A) and the primary escape time (C) of male mice in the 12 M group. (B) The average of (A). (D) The average of (C). (E) The representative images of p-tau in the 12 M mice cortex. Scale bar, 50 μm. (F-H) The statistical results of p-tau level in male and female mice cortex based on the data in (E). Mice were treated with rMSA 1.5 mg per gram of body weight or isometric saline every 3 weeks for 8 months. All graphs represent mean with SEM, with p values calculated by the two-tail t test. n, number of mice used for each analysis.

FIG. 4. rMSA treatment improved four parameters related to aging

(A-D) The level of free thiol (A), carbonyl (B), AGE (C), and Hcy (D) of rMSA and endogenous albumin from serum samples of mice at 1.5-, 12-, and 28 months of age. (E-H) The level of free thiol (E), carbonyl (F), AGE (G), and Hcy (H) of endogenous albumin of mice treated with body weight-adjusted dosage of rMSA or isometric saline in the 12 M group. (I-L) The level of free thiol (I), carbonyl (J), AGE (K), and hcy (L) of rMSA and endogenous albumin from serum samples of mice at 1.5-, 12-, and 20 months of age. All graphs represent mean with SEM, with p values calculated by the two-tail t test. n, number of mice used for each analysis.

FIG. 5. Expression and protein levels of albumin could return to normal in 21 days after the rMSA injection.

(A) Dynamic expression levels of the albumin gene in the liver determined by qRT-PCR after the injection of 20 mg rMSA per mouse (n=3). (B) Dynamic expression levels of the albumin gene in the liver determined by qRT-PCR after the injection with 50 mg rMSA per mouse (n=3). (C) Dynamic protein levels of the serum albumin within 1 day (upper left) and 2-21 days (upper right) after the injection with 20- or 50 mg rMSA per mouse (n=3). The table (bottom) shows the mean quantitative values. All graphs represent mean with SEM.

FIG. 6. Effects of rMSA injection on the total protein level and the albumin/globulin ratio

Dynamic total protein levels (A), total globulin levels (B), and the albumin/globulin ratio (C) within 1 day (upper left) and 2-21 days (upper right) after the injection with 20- or 50 mg rMSA per mouse (n=3). The table (bottom) shows the mean quantitative values. All graphs represent mean with SEM.

FIG. 7. Effects of rMSA injection on the major blood biochemical parameters

Dynamic changes of the major blood biochemical parameters (showed in line charts. GLU, glutamine; BUN, blood urea nitrogen; CHO, cholesterol; TG, triglyceride; LDH, lactic dehydrogenase; ALT, alanine aminotransferase; AST, aspartate aminotransferase; ALP, alkaline phosphatase; GGT, gamma-glutamyl transpeptidase). Tables (bottom) show the mean quantitative values. All graphs represent mean with SEM.

FIG. 8. Young and undamaged rMSA-treated mice showed a healthier appearance

Representative images of aged female mice injected with rMSA or saline. Kyphosis is arrowed.

FIG. 9. Effects of rMSA injection on body weight

Body weight of female and male mice in the (A) 6 M, (B) 3 M, and (C) 18 M group treated with 20 mg rMSA per mouse or isometric saline every 3 weeks. (D) Body weight of female and male mice in the 12 M group treated with 1.5 mg rMSA per gram of body weight or isometric saline every 3 weeks. All graphs represent mean with SEM.

FIG. 10. Representative motion trails in the Barnes maze test

(A) Representative motion trails of male mice treated with saline in the 12 M group.

(B) Representative motion trails of male mice treated with 1.5 mg rMSA per gram of body weight in the 12 M group.

FIG. 11. Intact protein mass spectrometry of rMSA

The molecular weight of rMSA was verified via Q-TOF mass spectroscopy.

FIG. 12. Comparison of the four parameters between the young and undamaged rHSA and blood-derived endogenous HSA.

(A-D) The level of free thiol (A), carbonyl (B), AGE (C), and Hcy (D) of the young and undamaged rHSA and blood-derived endogenous HSA. All graphs represent mean with SEM, with p values calculated by the two-tail t test.

### DETAILED DESCRIPTION OF THE INVENTION

The terms “a”, “an”, and “the” as used herein are intended to mean “one and more than one” or “at least one”, unless the context clearly suggests a singular meaning.

The term “and/or” as used herein are intended to include any and all possible combinations of one or more of the listed items.

The terms “comprises” and “comprising” as used herein are intended to indicate the presence of an element, component, feature, step etc., but not to exclude the presence of any other elements, components, features, steps etc. In the present invention, when it is mentioned that a product comprises certain components, it should be understood that it also recites the product which is composed only of these components.

The term “pharmaceutically acceptable carrier” encompasses any of the standard pharmaceutical carriers, buffers and excipients, including buffered saline solution, water, and emulsions (such as an oil/water or water/oil emulsion), and various types of wetting agents and/or adjuvants. Suitable pharmaceutical carriers and their formulations are described in Remington's Pharmaceutical Sciences (Mack Publishing Co., Easton, 19th ed. 1995). Preferred pharmaceutical carriers depend upon the intended mode of administration of the active ingredient agent.

In the present invention, unless otherwise specified, the sequence orientation is from 5′ to 3′.

As used herein, the term “human serum albumin” or “HSA” may be used interchangeably and shall be interpreted in a broad sense. The term “human serum albumin” or “HSA” includes wild type human serum albumin (see, e.g., Gene name: ALB; NCBI ID:213; UniProtKB-P02768), and any functional mutant thereof which comprises addition, deletion or substitution of one or several amino acid residues and maintains substantially the same biological functions of wild type HSA. The biological functions of HSA are well known in the art.

As used herein, the term “young” HSA and “young and undamaged” HSA have the same meaning and may be used interchangeably, which means the protein is in a fresh status and do not have extensive damages as compared to a specific HSA preparation (e.g. a HSA preparation obtained from an individual of 30 years old).

A skilled artisan will understand that the term “young and undamaged” HSA does not strictly require that there is absolutely no damage. It is very difficult, if not totally impossible, to prepare such perfect preparations. For the purpose of the present invention, a HSA preparation comprising limited damages is still acceptable.

“Young” HSA usually exhibits at least one, e.g. two or three, preferably all, of the following properties: (1) higher ratio of free thiol in Cys-34 residue, (2) lower level of advanced glycation end-product (AGE), (3) lower level of carbonylation, and (4) lower level of homocysteinylation. A younger HSA usually has a higher ratio of free thiol, lower level of AGE, carbonyl and homocysteine.

In some embodiments, “Young” HSA exhibits at least one, e.g. two or three, preferably all, of the following properties: (1) the ratio of free thiol in Cys-34 residue being greater than 50%, for example 70%, especially 80%, preferably 90%, more preferably greater than 95%, as determined by the Ellman's method; (2) the level of advanced glycation end-product (AGE) being lower than 60 μg/g protein, preferably 40 μg/g protein, more preferably lower than 30 μg/g protein, as determined by ELISA (CLOUD-CLONE Co., CEB353Ge); (3) the level of carbonyl is lower than 1.7 nmol/mg protein, preferably lower than 1.5 nmol/mg protein, as determined by Protein Carbonyl Content Assay Kit (Abeam); and (4) the level of homocysteine is lower than 5 nmol/g protein, preferably 3.5 nmol/g protein, more preferably lower than 2 nmol/g protein, as determined by ELISA (Jianglai, JL10022).

As used herein, the term “preparation” refers to a material prepared by a process. For example, a HSA preparation may refer to a HSA sample prepared from a particular individual, or from multiple individuals, which contains a population of human serum albumin molecules. “Preparation” may be a packaged commercial product, or a raw material prepared in the form of solution or lyophilized powder etc.

The Four Parameters and Measurement Thereof

Free Thiol: Wild type HSA (Gene name: ALB; NCBI ID:213; UniProtKB-P02768) contains one reduced cysteine residue (Cys-34 residue), which means one free thiol group per HSA molecule. The free thiol group of reduced Cys-34 residue can be oxidized in vitro and in vivo to an oxidized form.

The ratio of free thiols in HSA means:

$$\frac{The concentration of HSA with reduced Cys - 34 residue in a sample}{The concentration of HSA in a sample} \times 100 \%$$

Ellman's method: The absorbance increase at 412 nm after addition of DTNB.

AGE: Serum albumin is a plasmatic protein highly sensitive to glycation. This process, also known as the Maillard reaction, is a slow non-enzymatic reaction that initially involves attachment of glucose or derivatives with free amine groups of albumin to form reversibly a Schiff base product, leading to the formation of stable fructosamine residue (ketoamine) following Amadori rearrangement. This is the early glycation process: Schiff's base and fructosamines have been called early glycation adducts. The Amadori products could subsequently cyclize, forming pyranose or furanose carbohydrate adducts. Further modifications in these early stage glycation products, such as rearrangement, oxidation, polymerization and cleavage give rise to irreversible conjugates, called advanced glycation end products (AGE). Mass concentration of AGE was measured with an enzyme-linked immunosorbent assay (ELISA) kit according to manufacturer's instructions (CLOUD-CLONE Co., CEB353Ge). Results were normalized by HSA concentration per reaction and expressed as microgram AGE per gram protein.

Carbonyl: A carbonyl group contains a carbon atom that is double-bonded to an oxygen atom. Here, carbonylation means the formation of carbonyl groups on the amino acid residues of HSA. Protein Carbonyl Content Assay Kit (ab126287) is used to quantify the carbonyls in HSA. The method is based on the reaction of DNPH with protein carbonyls, which forms DNP hydrazones and can be quantified at 375 nm absorbance using a microplate reader. Carbonyl molarity was quantified using the Protein Carbonyl Content Assay Kit (Abeam, ab126287) according to the manual. Results were normalized by HSA concentration per reaction and expressed as nmol carbonyl per milligram protein.

Hcy: Homocysteine (Hcy) is metabolized from the essential dietary protein amino acid. Met and is considered to be a nonproteinogenic amino acid which is found to participate in protein post-translational modification (PTM). Hcy can be linked to protein via an isopeptide bond to lysine (Lys) residues (N-Hcy-protein) or via a disulfide bond to the Cys-34 residue (S-Hcy-protein). N-homocysteinylation is an emerging PTM that affect the structure and function of protein and causes protein damage. N-homocysteinylation is afforded by Hcy-thiolactone that arise only from Hcy. The molarity of Hcy can be determined quantitatively by ELISA and HPLC. In our study, Hcy molarity were measured by ELISA according to manufacturer's instructions (Jianglai, JL10022). Results were normalized by HSA concentration per reaction and expressed as nmol Hcy per gram protein.

Based on the findings from this research, young HSA will bring remarkable healthy advantages to a human subject who receives it.

The inventors creatively proposed the idea of defining the aging status of HSA by the following four parameters: (1) ratio of free thiol in Cys-34 residue, (2) level of advanced glycation end-product (AGE), (3) level of carbonylation, and (4) level of homocysteinylation.

In one aspect, the present invention provides a preparation of young and undamaged human serum albumin (HSA), wherein it exhibits at least one, e.g. two or three, preferably all, of the following properties: (1) higher ratio of free thiol in Cys-34 residue, (2) lower level of advanced glycation end-product (AGE), (3) lower level of carbonylation, and (4) lower level of homocysteinylation, as compared to an endogenous HSA preparation obtained from a young individual of human.

The term “young” individual of human usually refers to a human below age of 30, preferably below age of 18, more preferably below age of 3, or even below age of 1.

In an embodiment, there is provided a preparation of young and undamaged human serum albumin (HSA), wherein it exhibits at least one, preferably all, of the following properties: (1) the ratio of free thiol in Cys-34 residue being greater than 50%, for example 70%, especially 80%, preferably 90%, more preferably greater than 95%, as determined by the Ellman's method; (2) the level of advanced glycation end-product (AGE) being lower than 60 μg/g protein, preferably 40 μg/g protein, more preferably lower than 30 μg/g protein, as determined by ELISA (CLOUD-CLONE Co., CEB353Ge); (3) the level of carbonyl is lower than 1.7 nmol/mg protein, preferably lower than 1.5 nmol/mg protein, as determined by Protein Carbonyl Content Assay Kit; and (4) the level of homocysteine is lower than 5 nmol/g protein, preferably 3.5 nmol/g protein, more preferably lower than 2 nmol/g protein, as determined by ELISA (Jianglai, JL10022).

In wild type HSA, there is only one free thiol which is located in Cys-34 residue. A skilled artisan will easily understand that if the HSA is a functional mutant of wild type HSA and comprises a slightly different amino acid sequence, the Cys-34 residue might be located in a slightly different position than 34. The position is determined based on the amino acid sequence of wild type HSA.

In an embodiment, the ratio of free thiol in Cys-34 residue is determined by the Ellman's method. Preferably, the ratio of free thiol in Cys-34 residue is greater than 70%, especially 80%, preferably 90%, more preferably greater than 95%.

In an embodiment, the level of AGE is by ELISA (CLOUD-CLONE Co., CEB353Ge). Preferably, the level of AGE is lower than 60 μg/g protein, preferably 40 μg/g protein, more preferably 30 μg/g protein, as determined by ELISA.

In an embodiment, the level of carbonyl is determined by Protein Carbonyl Content Assay Kit (Abcam). Preferably, the level of carbonyl is lower than 1.7 nmol/mg protein, preferably lower than 1.5 nmol/mg protein.

In an embodiment, the level of homocysteine is determined by ELISA (Jianglai, JL10022). Preferably, the level of homocysteine is lower than 5 nmol/g protein, preferably 3.5 nmol/g protein, more preferably lower than 2 nmol/g protein.

A skilled artisan will understand that the abovementioned four parameters may be tested by other commonly recognized methods and the numerical results of a parameter tested from the same sample by different methods might be different. A skilled artisan will be able to compare the results tested from different methods with the results obtained by the methods used in this invention by limited amount of experiments.

In a preferred embodiment, the ratio of free thiol in Cys-34 residue is greater than 80%, the level of AGE is lower than 30 μg/g protein, the level of carbonyl is lower than 1.5 nmol/mg protein, the level of homocysteine is lower than 2 nmol/g protein.

The preparation of young HSA may be produced recombinantly or purified from plasma. Preferably, the HSA preparation is produced recombinantly.

In another aspect, there is provided use of a preparation of young and undamaged human serum albumin (HSA) in extending lifespan and/or anti-aging in a subject, wherein it exhibits at least one, preferably all, of the following properties: (1) higher ratio of free thiol in Cys-34 residue, (2) lower level of advanced glycation end-product (AGE), (3) lower level of carbonylation, and (4) lower level of homocysteinylation, as compared to an endogenous HSA preparation obtained from said subject.

In this situation, the subject will obtain healthy benefits by receiving a “younger” HSA preparation as compared to an endogenous HSA preparation obtained from himself. For example, if the subject is 70 years old, then a HSA preparation obtained from a subject, or at an aging status, of below 70 years old, e.g. 50, 40 or 30, will be effective.

The aging status of a HSA preparation may be evaluated by the abovementioned four parameters. If several, or preferably all, of the four parameters of a particular HSA preparation are better than those obtained from a subject of a specific age, e.g. 30, then this preparation is decided to be at an aging status younger than 30 years old.

There is provided a method of extending lifespan and/or anti-aging in a subject, comprising the step of administering to the subject an effective amount of a preparation of young and undamaged human serum albumin (HSA), wherein the preparation exhibits at least one, preferably all, of the following properties: (1) higher ratio of free thiol in Cys-34 residue, (2) lower level of advanced glycation end-product (AGE), (3) lower level of carbonylation, and (4) lower level of homocysteinylation, as compared to an endogenous HSA preparation obtained from said subject.

The subject may be a healthy individual or a patient suffering from a disease or injury.

In an embodiment, the preparation exhibits at least one, preferably all, of the following properties: (1) the ratio of free thiol in Cys-34 residue being greater than 50%, for example 70%, especially 80%, preferably 90%, more preferably greater than 95%, as determined by the Ellman's method; (2) the level of advanced glycation end-product (AGE) being lower than 60 μg/g protein, preferably 40 μg/g protein, more preferably lower than 30 μg/g protein, as determined by ELISA (CLOUD-CLONE Co., CEB353Ge); (3) the level of carbonyl is lower than 1.7 nmol/mg protein, preferably lower than 1.5 nmol/mg protein, as determined by Protein Carbonyl Content Assay Kit; and (4) the level of homocysteine is lower than 5 nmol/g protein, preferably 3.5 nmol/g protein, more preferably lower than 2 nmol/g protein, as determined by ELISA (Jianglai, JL10022).

There is provided a pharmaceutical composition, comprising the young HSA as defined herein and a pharmaceutically acceptable carrier.

In another aspect, there is provided a method for monitoring the change of aging status of a subject, comprising:

obtaining a sample of human serum albumin (HSA) from the subject;

testing in the sample at least one, preferably all, of the following four parameters of HSA: (1) ratio of free thiol in Cys-34 residue, (2) level of advanced glycation end-product (AGE), (3) level of carbonylation, and (4) level of homocysteinylation;

if the ratio of free thiol in Cys-34 residue increases and/or the level of AGE, the level of carbonylation and/or the level of homocysteinylation decreases, as compared to the data from an earlier test from the same subject, it is determined that the aging status of the subject is improved.

In another aspect, there is provided a method of improving the function of skeletal muscle and/or improving the cognitive ability in a subject, comprising the step of administering to the subject an effective amount of a preparation of young and undamaged human serum albumin (HSA) as described above.

To avoid immune response, all animal studies were carried out with rMSA (recombinant murine serum albumin), which is young and undamaged by the four parameters as compared with endogenous MSA (murine serum albumin) from mice at different ages. Based on the improvement of rMSA in the longevity and healthspan in mice, it is reasonable to expect young and undamaged rHSA would get similar effect in humans.

### EXAMPLES

Ludwig et al reported the extended lifespan of older rats by younger rats in the parabiosis model for the first time in 1972¹. Egerman group and Villeda group respectively found that the muscle strength and cognitive ability of old mice were improved after the parabiosis surgery with young mice^{2,3}, which suggest that the “mystery” of aging might exist in blood. As a major part of the circulatory system, blood may modulate aging process by affecting cells and tissues throughout the body. It has been shown that human plasma proteomes change with age⁴. It is believed that aging is at least partially caused by the continuous accumulation of structural changes or damages of macromolecules⁵⁻⁷. Firstly, it has been reported that oxidative stress plays a vital role in aging^{8,9}, which is associated with the destruction of proteins by reactive oxygen species (ROS). In human beings, the plasma thiol/disulfide redox state (REDST) becomes more pro-oxidative with age^{10,11}. Secondly, the importance of non-enzymatic protein glycation has been noticed in aging research¹²⁻¹⁴. The level of plasma advanced glycation end-product (AGE) is elevated in older people¹⁵. Thirdly, in oxidative environments, carbonyls are also formed especially on the side chains of Pro, Arg, Lys and Thr residues in proteins^{16,17}. Importantly, the concentrations of carbonyls increase as people get older¹⁸. Fourthly, it has been widely reported that homocysteine (Hcy) increases with age and is associated with age-related degenerative disorders¹⁹⁻²². Hcy has been found to covalently bind with proteins thus disturb their normal functions^{23,24}. Altogether, damages or unnecessary modifications of plasma proteins increase with age. It is plausible to propose that reducing damages or unnecessary modifications of plasma proteins may increase the longevity even reverse the aging process.

Human serum albumin (HSA) is the most abundant protein in blood plasma with a serum half-life of about 21 days²⁵. It not only maintains a stable plasma colloid osmotic pressure, but also is an important carrier²⁶. These functions of HSA endow it with various clinical applications²⁶.

In addition to the basic properties, the oxidation and glycation of HSA are widely reported to be related to some pathological conditions. The single free thiol in Cys-34 residue of HSA has been proposed to account for approximately 80% of the total free thiols in plasma, which provides the antioxidant function^{27,28}. It was noticed that oxidation of the HSA thiol is intimately linked with aging and age-related diseases²⁹. Leto et al found that not only the concentrations of free thiols and HSA but also the ratio of free thiol to HSA decreased with age, which was due to the changes in the redox potential during aging³⁰. Era et al suggested that HSA could function as a redox buffer to maintain a constant redox potential of the extracellular fluids³¹. The fraction of reduced albumin in young male subjects was larger than that of elderly subjects. Besides the thiol oxidation in HSA, elevated carbonyl levels have been found to be related to aging and varieties of diseases³²⁻³⁴. The glycation of albumin is another important factor found to be involved in aging and senile diseases. Serum albumin, as a long-lived protein, accumulates AGE with age in vivo³⁵. It is widely reported that glycation impairs normal functions of albumin and can induce inflammatory responses, which is connected with aging and the progression of serious diseases^{35,36}. In addition, HSA is a major target for Hcy modification, thus it can efficiently protect other proteins from the toxicity of Hcy^{23,37}. Therefore, treatment of freshly prepared recombinant serum albumin with nearly no damages or unnecessary modifications is most likely to extend lifespan and healthspan. Here we report that young and undamaged recombinant mouse serum albumin (rMSA)-treated groups in natural aging mouse model remarkably obtained significantly extended lifespan with increased skeletal muscle strength and cognitive ability compared with saline-treated groups.

### Example 1: rMSA Treatment Increased the Longevity in Mice

In order to verify whether rMSA treatment can extend the lifespan of mice, C57BL/6N mice at different ages were chosen for natural aging models. Mice in the first cohort (6 M) were 6-month age, and were i.v. injected with 20 mg rMSA or isometric saline every 3 weeks until death. qRT-PCR and blood biochemical analyses showed that both mRNA and protein levels of albumin underwent slight fluctuations before returning to normal within 8 days after the first injection (FIG. 5). Major blood biochemical parameters remained constant in normal levels (FIG. 6, 7).

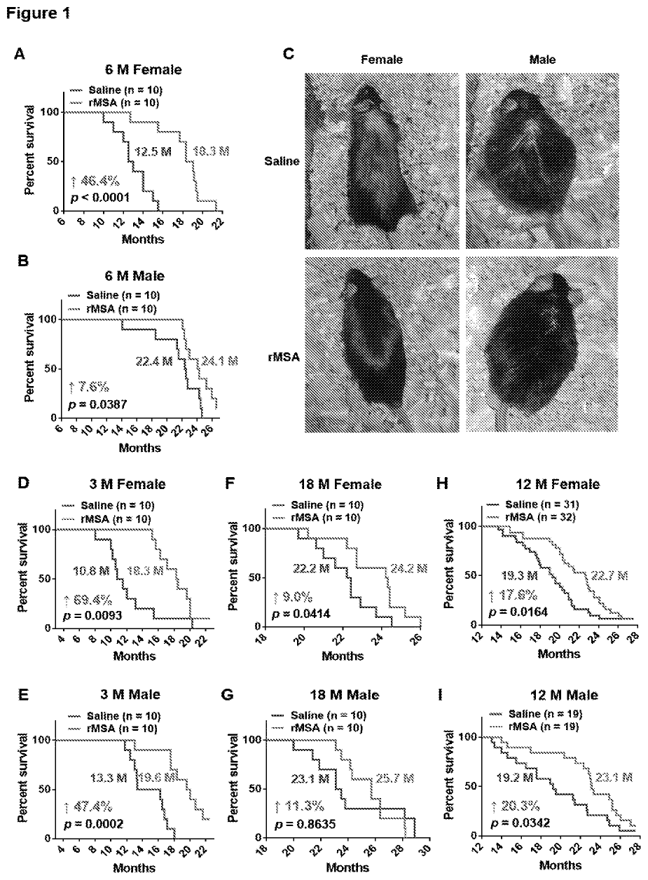

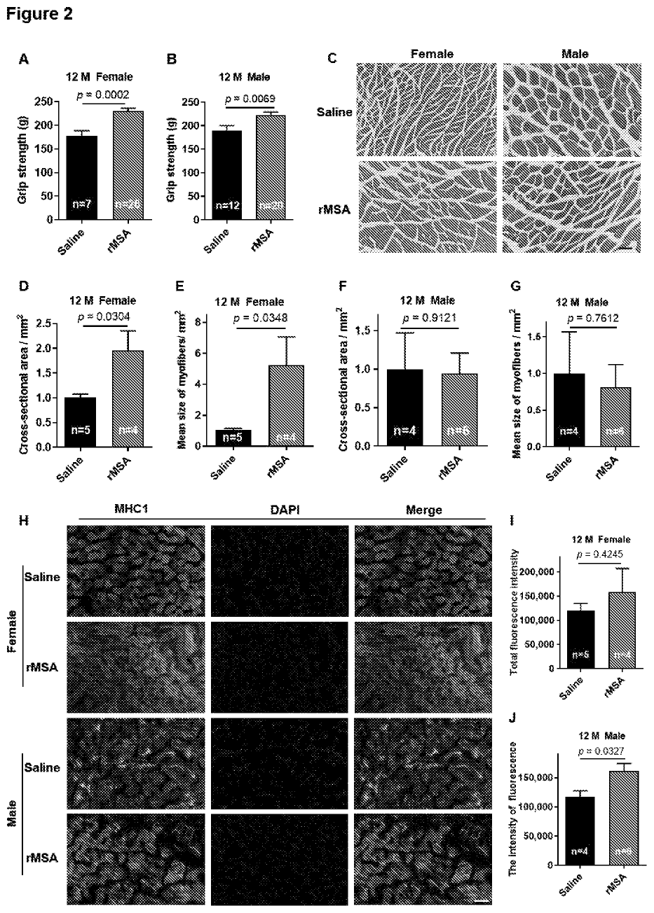

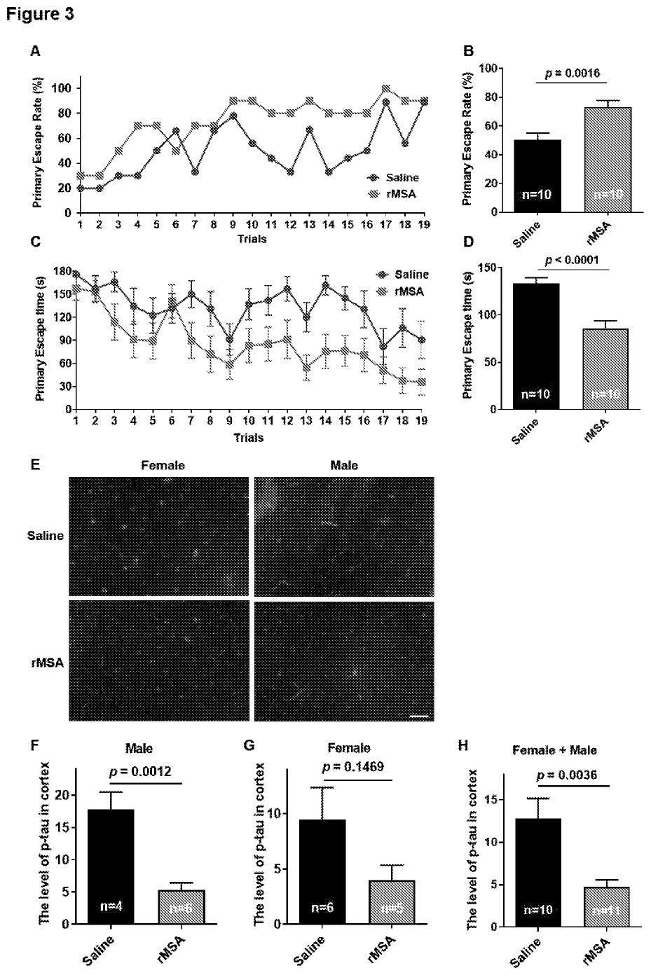

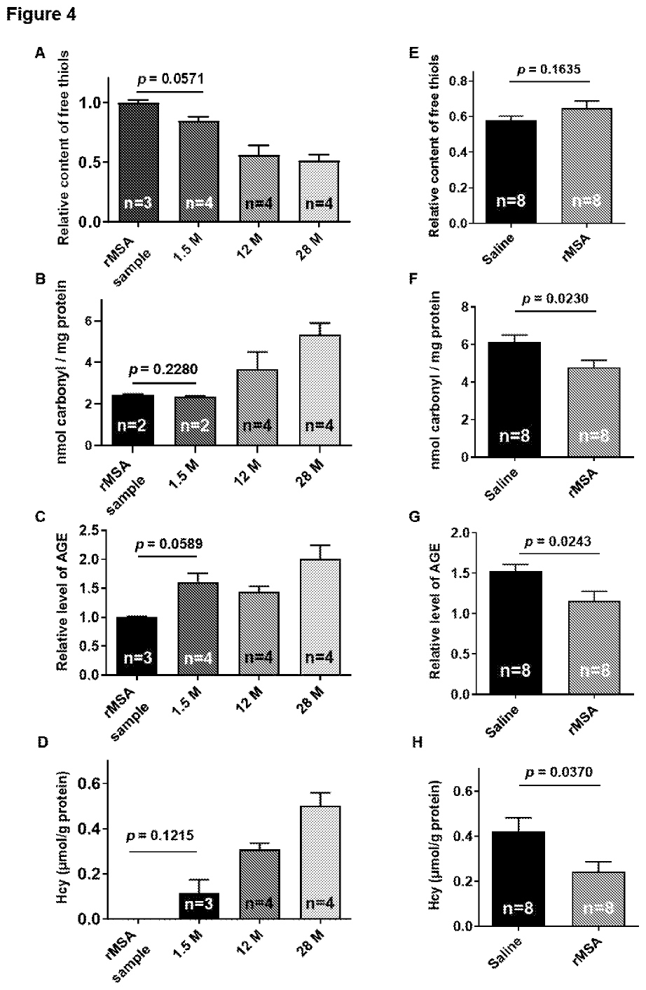

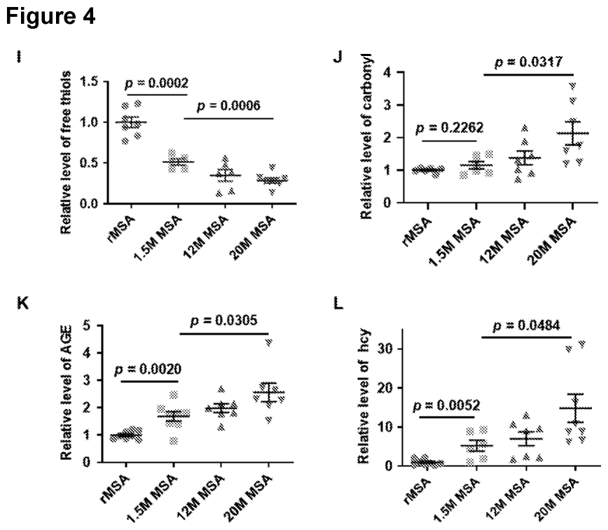

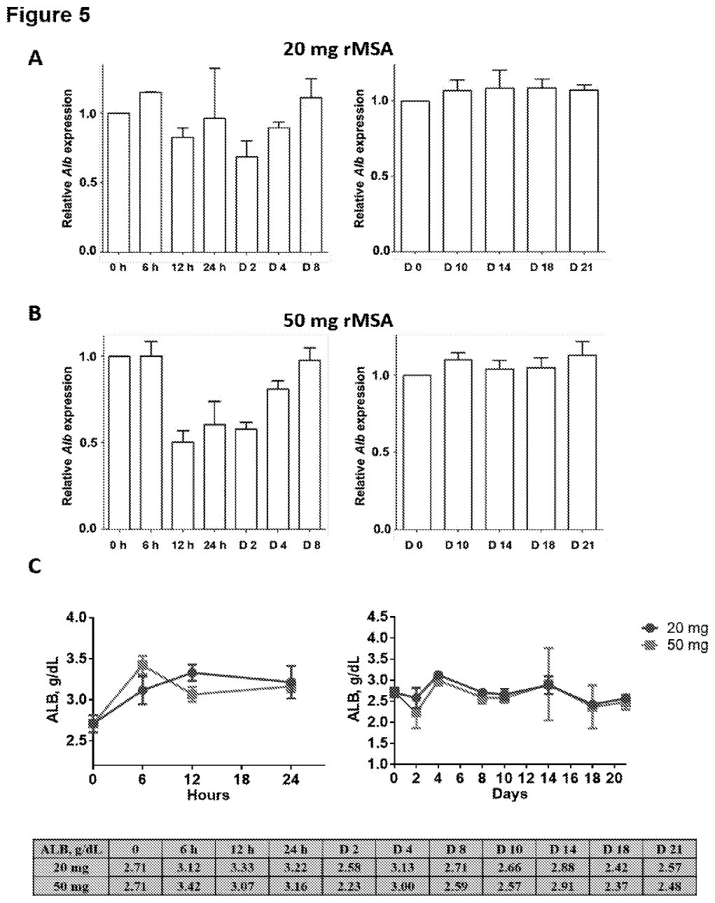

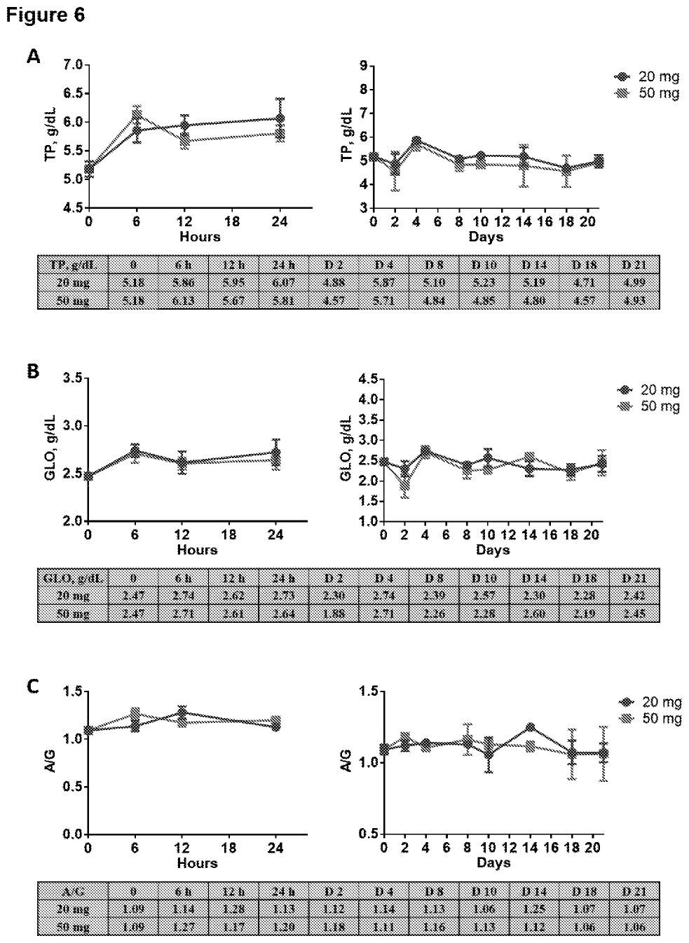

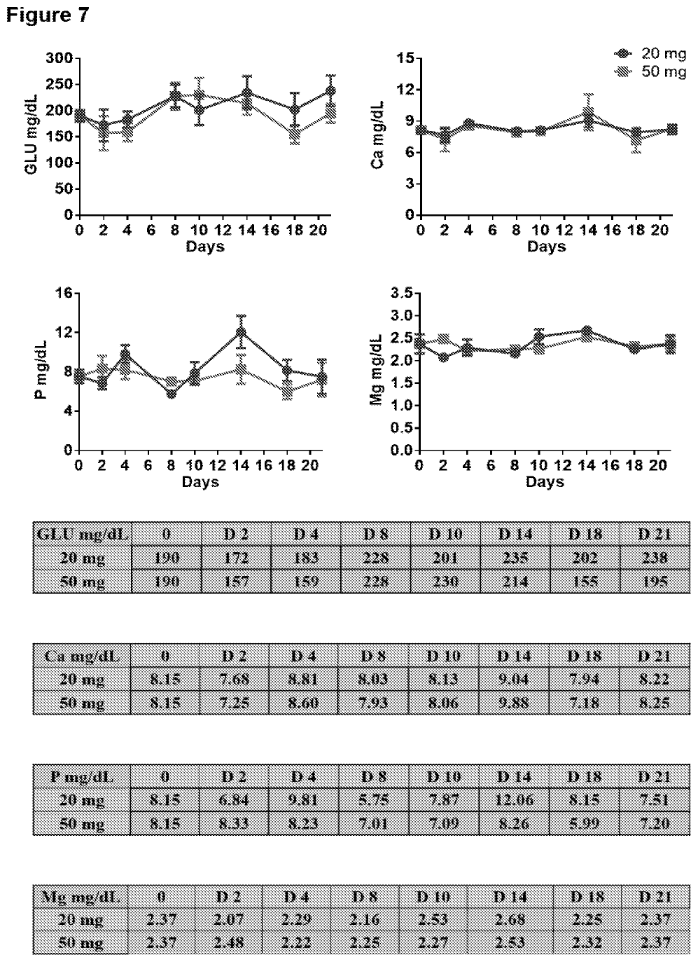

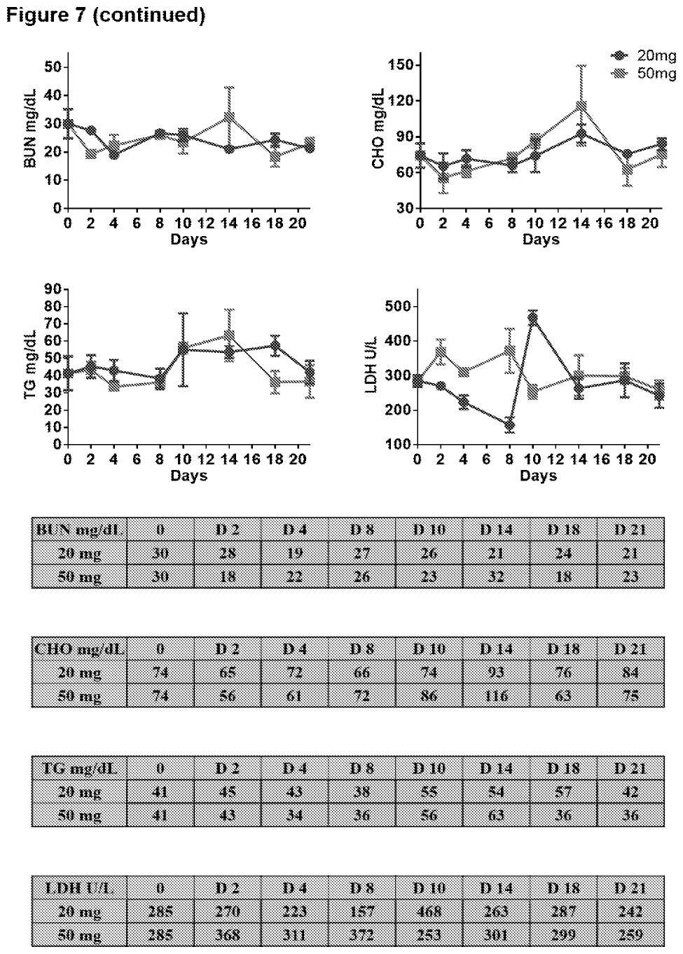

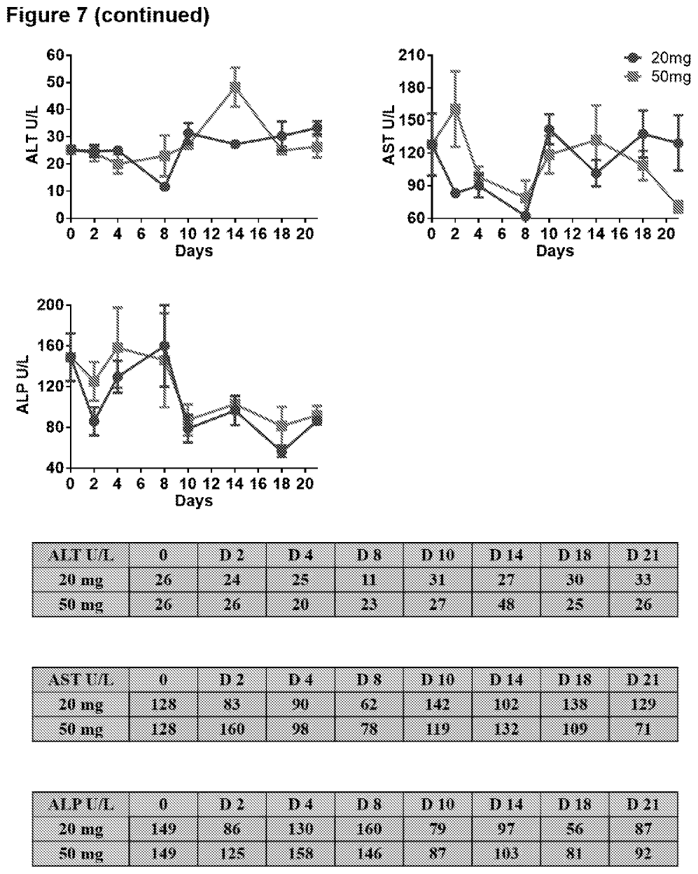

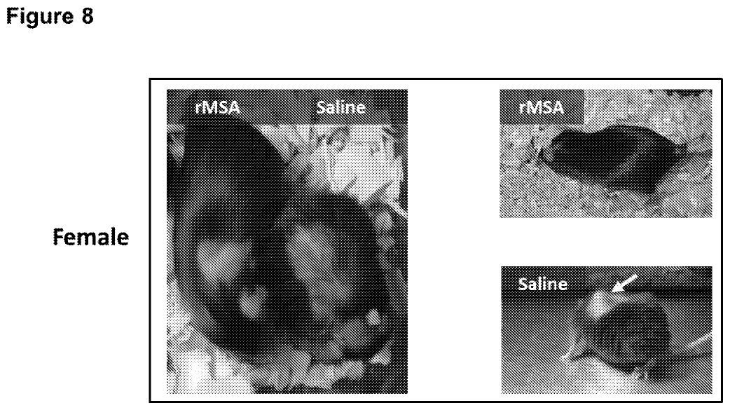

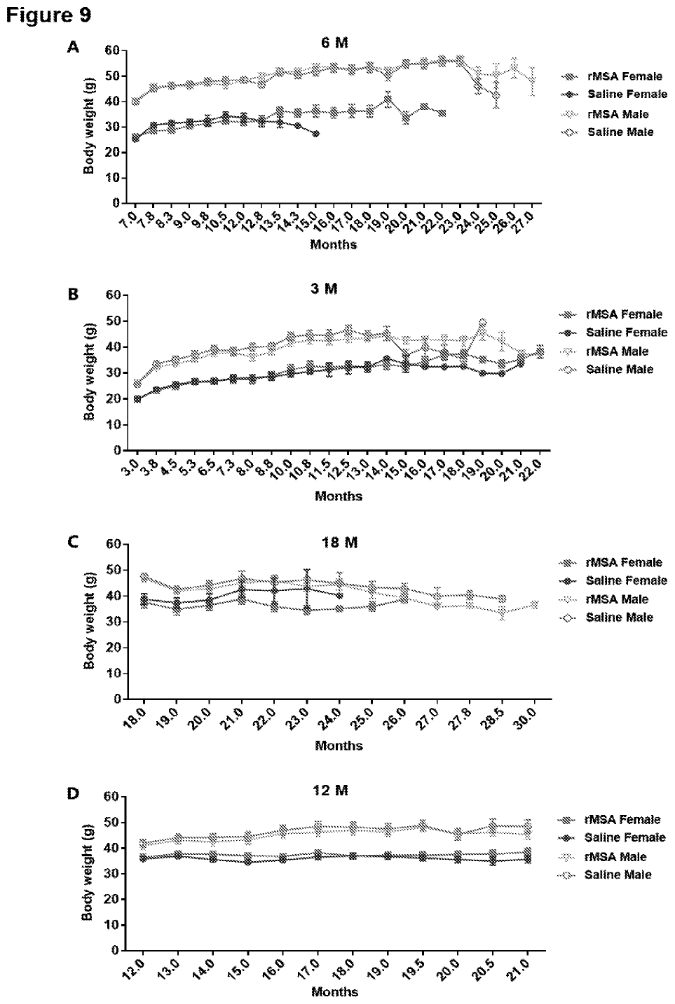

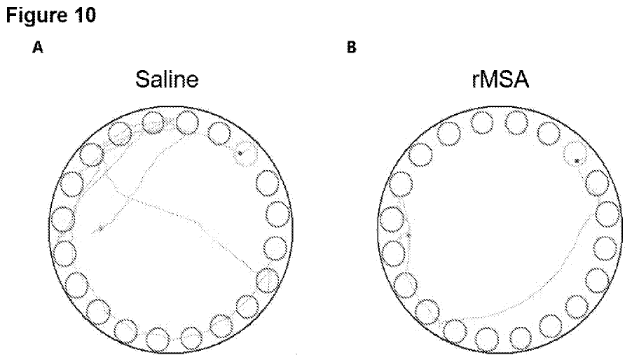

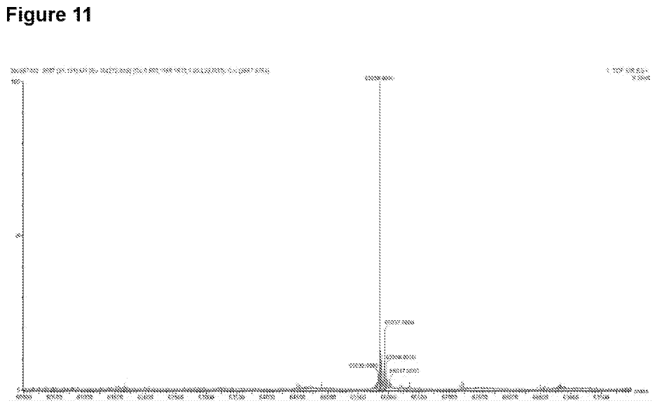

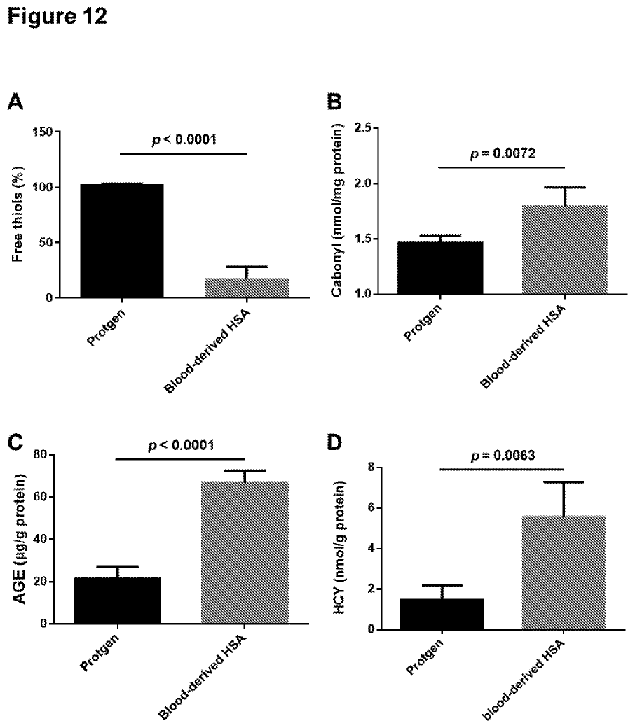

Survival curves for each gender showed that the median lifespans of rMSA-treated mice were respectively increased by 46.4% (female, 5.8 months increased, p<0.0001) and 7.6% (male, 1.7 months increased, p=0.0387, see discussion below) (FIG. 1A, B). Changes in the appearance of both sexes were observed when the median lifespan was reached. Interestingly, mice treated with rMSA had glossier and thicker fur than saline-treated mice (FIG. 1C). In addition, it was observed that kyphosis was more severe in the saline-treated group than in the rMSA-treated group (FIG. 8). These phenomena suggest that rMSA treatment is safe for long-term use and can extend the lifespan of C57BL/6N mice in the 6 M group.

We proposed that the younger the mice started to be treated with rMSA, the more profound effects could be achieved. Thus 3-month-old mice were chosen in the second cohort (3 M) using the same dosage as the 6 M group. Astonishingly, the median lifespans of rMSA-treated female and male mice were increased by 69.4% (female, 7.5 months increased, p=0.0093) and 47.4% (male, 6.3 months increased, p=0.0002), respectively, when compared with saline-treated group (FIG. 1D, E).

Similarly, we wondered if rMSA treatment has any effect on old mice. 18-month-old mice were chosen in the third cohort (18 M). The median lifespans of rMSA-treated mice in the 18 M group were improved for both genders, 9.0% for females (2.0 months increased, p=0.0414) and 11.3% for males (2.6 months increased, p=0.8635) (FIG. 1F, G). Moreover, rMSA has no effect on the body weight in all groups (FIG. 9).

We noticed that in the 3 M and the 6 M groups, the lifespan extension of male mice was not as dramatic as that of female mice treated with the same amount of rMSA, which was not observed in the 18 M group, probably due to the large differences in the body weight between female and male mice in the 3 M and the 6 M groups, but not in the 18 M group (FIG. 9). Therefore, we proposed that the amount of rMSA should be adjusted by body weight to obtain an equivalent dosage.

The dosage of rMSA for each mouse was adjusted from 20 mg to 1.5 mg per gram of body weight every 3 weeks. 12-month-old mice were chosen in the fourth cohort (12 M) for the injection of body weight-adjusted dosage. The lifespans of rMSA-treated mice in the 12 M group improved significantly, 17.6% for females (3.4 months increased, p=0.0164) and 20.3% for males (3.9 months increased, p=0.0342) (FIG. 1H, I), which indicate that the injection of body weight-adjusted dosage can reduce the difference in lifespan extension between two genders.

### Example 2: rMSA Enhanced the Function of Skeletal Muscle in Mice

The striking elongation in mice lifespan triggered us to further explore whether the healthspan could also be improved. As the dysfunction in skeletal muscle was commonly observed during aging, we first detected the changes of grip strength in mice treated with body weight-adjusted dosage of rMSA or isometric saline for 8 months in the 12 M group.

rMSA-treated mice in the 12 M group exhibited significantly increased forelimb grip strength from 177.9 g to 230.5 g (29.6% increased, p=0.0002) in females and from 189.6 g to 222.5 g (17.4% increased, p=0.0069) in males, as compared to saline-treated mice (FIG. 2A, B).

In order to evaluate the effect of rMSA injection on the in vivo skeletal muscle size and quality, we further performed histological analysis on gastrocnemius muscle (FIG. 2C). We found that the cross-sectional area and the mean size of myofibers in rMSA-treated female mice were significantly increased respectively (cross-sectional area 94.6% increased, p=0.0304; mean size 405.2% increased, p=0.0348) than those in the saline group (FIG. 2D, E). However, similar phenomenon was not observed in male mice (FIG. 2F, G). We next investigated the expression level of slow myosin heavy chain I (MHC1) in rMSA and saline-treated group, another important parameter to evaluate the muscle strength (FIG. 2H). Male mice treated with rMSA presented significantly more slow MHC1 positive fibers than saline-treated mice (37.7% increased, p=0.0327), while similar results were not obtained in female mice (FIG. 2I, J). Taken together, it was demonstrated that rMSA treatment enhanced the size of gastrocnemius fibers in female mice, and increased the level of slow MHC1 in male mice, but had little effect on the number and circumference of fibers.

### Example 3: The Cognitive Ability of Mice was Improved by Treatment with rMSA

We next investigated the effect of rMSA on aging-related impairment of memory using the Barnes Maze tests. Male mice in the 12 M group were chosen and were treated with rMSA or saline for 8 months. rMSA-treated group exhibited a dramatic increase in the primary escape rate (73.2% v.s. 50.2%, 23.0% increased, p=0.0016) compared to that of the saline-treated group (FIG. 3A, B). Meanwhile, the rMSA-treated male mice displayed significantly reduced primary escape time (85.8 sec v.s. 133.4 sec, 47.6 sec faster, p<0.0001) than the saline-treated mice (FIGS. 3C and D, FIGS. 10A and B). All these results demonstrated that rMSA treatment significantly improved the ability of spatial learning and memory in aging mice.

We then evaluated the histological changes associated with the cognitive ability using these groups of mice. Excitingly, the results of immunofluorescence (IF) staining in the cortex showed that the level of p-tau was significantly decreased by rMSA treatment in male mice than that of the saline group (70.1% decreased, p=0.0012) (FIG. 3E, F). However, there was no significant discrepancy in female groups, though the level of p-tau in rMSA-treated mice was lower than that of the saline-treated mice (57.8% decreased, p=0.1469, FIGS. 3E, G). In sum, injection of rMSA could decrease the p-tau level of mice (63.2% decreased, p=0.0036, FIG. 3H), especially male mice.

### Example 4: rMSA Treatment Improved Four Parameters Related to Aging

Previous studies have demonstrated that aging was closely related to increasing levels of protein damages, including oxidation of free thiol, carbonylation, AGE formation, and homocysteinylation, total of four parameters. The endogenous albumin from serum samples of mice at 1.5-, 12-, and 28 months of age was purified for comparison. During the aging process, serum albumin undergoes a series of changes in the four parameters: decreased level of free thiol and increased levels of carbonyl, AGE, and Hcy. The rMSA used in this study is even younger and less damaged than endogenous serum albumin from the young mice even at 1.5 months of age. The rMSA contains more free thiols (18.1% increased, p=0.0571), equivalent level of carbonyl, less AGE (37.7% decreased, p=0.0589), less Hcy (not detected in rMSA, p=0.1215) (FIG. 4A-D), which endows rMSA to offer more protection against unnecessary modifications and damages.

In order to explore how young and undamaged rMSA improved the lifespan and healthspan of mice, 12-month-old mice were treated with 1.5 mg rMSA per gram of body weight or isometric saline every 3 weeks for 8 months. All serum samples were collected 21 days after the last injection. The albumin from the rMSA-treated mice contained more free thiols (11.6% increased, p=0.1635), much lower levels of carbonyl (22.1% decreased, p=0.0230), AGE (24.4% decreased, p=0.0243), and homocysteine (42.6% decreased, p=0.0370) than that of the saline-treated mice (FIG. 4E-H).

An additional test was conducted with larger sample size for the comparison of the four parameters, in which more dramatic significance was obtained. When compared with the endogenous serum albumin from 1.5-month-old mice, the exogenous rMSA contains more free thiols (95.5% increased, p=0.0002), less carbonyl (13.0% decreased, p=0.2262), less AGE (40.6% decreased, p=0.0020), and less Hcy (80.9% decreased, p=0.0052) (FIG. 4I-L), which endows rMSA to offer more protection against unnecessary modifications and damages.

Taken together, young and undamaged rMSA provides a powerful protective function against oxidation of free thiol, carbonylation, AGE formation, and homocysteinylation.

### Example 5: Young and Undamaged rHSA is Different from Endogenous HSA in Four Parameters Related to Aging

In order to figure out the difference between the young and undamaged rHSA and endogenous HSA, 3 blood-derived endogenous HSA products from independent manufacturers were purchased for comparison studies, in which free thiol, carbonyl, AGE, and homocysteine were tested as described below.

Firstly, the Ellman's method was used to determine the absolute amount of free thiol which was divided by the amount of HSA (a HSA molecule theoretically has only one free thiol) to obtain the percent residual free thiol content. It was shown that rHSA (Protgen) contained intact free thiol while that of blood-derived HSA was damaged seriously (102.8% v.s. 17.6%, p<0.0001) (FIG. 12A).

Secondly, carbonyl molarity was quantified using the Protein Carbonyl Content Assay Kit (Abcam, ab126287) according to the manual. Results were normalized by HSA concentration per reaction and expressed as nmol carbonyl per milligram protein. We found that the carbonyl level of rHSA (Protgen) was lower than that of blood-derived HSA (1.46 v.s. 1.80 nmol/mg protein, p=0.0072) (FIG. 12B).

Thirdly, mass concentration of AGE was measured with an enzyme-linked immunosorbent assay (ELISA) kit according to manufacturer's instructions (CLOUD-CLONE Co., CEB353Ge). Results were normalized by HSA concentration per reaction and expressed as microgram AGE per gram protein. Obviously, rHSA (Protgen) contained less AGE than blood-derived HSA (21.4 v.s. 66.8 μg/g protein, p<0.0001) (FIG. 12C).

Forthly, homocysteine (Hcy) molarity were measured by ELISA according to manufacturer's instructions (Jianglai, JL10022). Results were normalized by HSA concentration per reaction and expressed as nmol Hcy per gram protein. It was shown that the Hcy level of rHSA (Protgen) was much lower than that of blood-derived HSA (1.46 v.s. 5.56 nmol/g protein, p=0.0063) (FIG. 12D).

| | | | | | Average of |
|---|---|---|---|---|---|
| Parameters | rHSA | Product 1 | Product 2 | Product 3 | Products 1-3 |
| Free thiol | 102.8 | 9.65 | 31.7 | 11.3 | 17.6 |
| (%) | | | | | |
| Carbonyl | 1.46 | 1.77 | 1.71 | 1.91 | 1.80 |
| (nmol/mg | | | | | |
| protein) | | | | | |
| AGE (μg/g | 21.4 | 62.5 | 71.8 | 66.1 | 66.8 |
| protein) | | | | | |
| Hcy (nmol/g | 1.46 | 5.35 | 6.34 | 5.00 | 5.56 |
| protein) | | | | | |

### Discussion

Part 1. Lifespan

In the present invention, the lifespan extension percentage of rMSA-treatment in the 3 M, the 6 M, and the 18 M groups are 69.4%, 46.4%, and 9.0% for female; 47.4%, 7.6%, and 11.3% for male, respectively, and healthspan can also be significantly increased through long-term rMSA treatments. These results suggest that the optimal effect should be obtained when the treatment starts as early as possible.

However, the lifespan extension percentage of male mice in the 3 M and the 6 M groups were not as dramatic as those of female mice under the same amount of rMSA. This is probably because the body weights of male mice are much heavier than those of female mice during that age. Subsequently, we adjusted the amount of rMSA based on body weight to inject an equivalent dosage on each mouse, and obtained consistent results in both genders in the 12 M group. Therefore, the body weight-adjusted dosage is critical for optimal effects.

Part 2. Healthspan

As to healthspan, rMSA treatment with body weight-adjusted dosage resulted in a significant increase in the forelimb grip strength of mice in the 12 M group. The improvement of the grip strength by rMSA treatment was coincident with that of the lifespan, demonstrating that rMSA most likely regulates both the lifespan and the healthspan based on the same fundamental principles.

Furthermore, we found that rMSA improved the function of gastrocnemius muscle by increasing the proportion of type I fibers in male mice and enlarging the size of myofibers in female mice. One explanation for these differences between male and female mice might be the variance of hormones and metabolic mechanisms.

rMSA injection can effectively improve the abilities of spatial learning and memory of mice as tested by Barnes Maze, and remarkably reduce the p-tau levels in the cortex of old mice. Effects of rMSA injection on memory improvement are being further verified in neurodegenerative disease models in our ongoing projects.

Part 3. Four Parameters

Based on our analyses, it is plausible to propose that young and undamaged rMSA provides a powerful protective function against oxidation of free thiol, carbonylation, AGE formation, and homocysteinylation.

Here in the examples, “young” means that the rMSA is much fresher than the endogenous albumin from young mice at the age of only 1.5 months analyzed by the 4 parameters (free thiol, carbonyl, AGE, and homocysteine). “Undamaged” theoretically means intact free thiol, no AGE, no carbonylation, and no homocysteinylation. In reality, due to the preparation process and detection methods, it is almost impossible to get such perfect sample.

As such, compared with endogenous serum albumin from the young mice even at 1.5 months of age, rMSA contains more free thiols, equivalent level of carbonyl, less AGE and homocysteine. We need to emphasize here that no other damage was observed in our samples, because the molecular weight measured by mass spectrum (FIG. 11) is exactly the same as the theoretically calculated value³⁸. In sum, rMSA used in this study is not only “young” but also almost “undamaged”.

We realized that effects of rMSA and endogenous albumin on the longevity of mice should be compared in parallel. In order to perform this experiment, endogenous albumin should be prepared from mice at different ages ranging from very young (3 months) to very old (29 months, according to our data), whenever rMSA was used. However, endogenous mouse serum albumin of sufficient purity is not commercial available. Moreover, at least 100,000 mice at different ages were needed to purify sufficient amount of albumin at a purity greater than 99%, which is unethical.

A clinical trial whose purpose was to evaluate the beneficial effects of infusions of plasma from young donors (16-25 years old) to older adults (≥35 years old) was initiated in 2016 in the USA, but no result has been released so far (ClinicalTrials.gov Identifier: NCT02803554). Most recently, Conboy group reported rejuvenation of muscle, liver, and hippocampus of mice by exchanging old blood plasma with saline containing 5% endogenous albumin³⁹. However, neither of these studies used recombinant serum albumin, which makes the results incomparable.

Part 4. Optimized Extension of Lifespan and Healthspan & Other Proteins

In 2014, Wyss-Coray group reported that plasma from young mice can improve the learning and memory of old mice. Since albumin occupies about 50% of total plasma proteins, it most likely plays the most important role in this process, which was exactly what we found here. In order to achieve the maximal effect of rMSA on longevity, a variety of measures including optimal dosage, frequency, and drug delivery methods are being investigated. We predict that the lifespan and healthspan will be extended more dramatically by rMSA if early and sustained-release of high dosages are used given tolerance and safety are assured.

We further predict that the concept of young and undamaged albumin increasing the longevity can also be applied to any other proteins such as immunoglobulins, fibrinogen, transferrin, transthyretin, and haptoglobin which are major plasma proteins.

It was well documented that the 4 parameters including free thiol, carbonyl, AGE, and homocysteine are closely related to various diseases such as diabetes mellitus, cardiovascular diseases, adiposity, and Alzheimer's disease^{20,24,40-43}.

It will be remarkable to see that a single young and undamaged protein (either recombinant or non-recombinant) HSA can increase the longevity of human beings, which will be initiated in the near future. If so, the combination of young and undamaged major plasma proteins can further increase the longevity. Ideally, all of the young and undamaged plasma proteins altogether can increase the longevity to the largest extent.

Methods

Mice and Drug Treatments

C57BL/6N mice were purchased from Beijing Vital River Laboratory Animal Technology Co., Ltd. (a distributor of Charles River Laboratories in China). The mice transport stress syndrome was carefully avoided during the transportation to the Laboratory Animal Research Center, Tsinghua University (THU-LARC). All mice were quarantined for one month to guarantee the adaptation to the new environment and carried out quality inspection. Animals were kept in a pathogen-free barrier environment with a 12-h dark-light circle. Room temperature was maintained at 23° C. After arrival, mice were fed with irradiation-sterilized JAX-standard breeder chow (SHOOBREE®, Xietong Pharmaceutical Bio-technology Co., Ltd., 1010058) and sterilized water during the entire study.

When the mice reached indicated ages (3-, 6-, 12-, and 18 months), they were divided into rMSA- or saline-treated group randomly. One kilogram rMSA was kindly supplied by Shenzhen Protgen, Ltd. The quality of GMP-grade rMSA, expressed by Pichia pastoris, was strictly controlled to ensure that the purity is greater than 99%. Most importantly, host cell proteins (HCPs) were less than 1 μg/g rMSA by ELISA, which means our rMSA is almost free of HCPs.

125 mg/mL of rMSA dissolved in saline was i.v. injected slowly. Mice were weighed before each injection to calculate the dosage, with saline served as the negative control. Mice were injected with 20 mg rMSA per mouse or 1.5 mg rMSA per gram of mouse body weight and isometric saline every 3 weeks as indicated. All animal studies were approved by the Institutional Animal Care and Use Committee of Tsinghua University (Beijing, China).

Protein Levels Determination

To determine the blood biochemical parameters, blood samples were collected from mouse tail vein or orbital sinus after Avertin® (Tribromoethanol, Sigma-Aldrich, T48402) intraperitoneal injection (400 mg/kg) for anesthesia. Serum samples were collected after centrifugation at 1,000×g for 20 min at 4° C. To collect plasma samples, heparin sodium salt is added to the fresh blood samples (20 units/mL blood, Sigma-Aldrich, H3149) to prevent blood clotting followed by centrifugation at 1,000×g for 30 min at 4° C. Major blood biochemical parameters of serum samples were determined with an automatic biochemistry analyzer (Olympus AU 400).

To determine the expression level of albumin, mice were euthanized using carbon dioxide after anesthesia. Liver tissue samples were quickly removed and homogenized. The total RNA from the homogenate was isolated using TRIzol Reagent (Invitrogen, 15596026) and converted into cDNA using the First Strand cDNA Synthesis Kit (Fermentas, K1622). Quantitative RT-PCR (qRT-PCR) was performed using the TransStart® Top Green qPCR SuperMix (TransGen Biotech Co., AQ131). Relative quantitation was analyzed using the 2\hat{ }-ΔΔCt method. Glyceraldehyde 3-phosphate dehydrogenase (GAPDH) was used as an internal control. Independent experiments were repeated in triplicates. The following primers were used: Alb forward 5′-TGCTTTTCCAGGGGTGTGTT, reverse 5′-TTACTTCCTGCACTAATTTGGCA; Gapdh forward 5′-GTTGTCTCCTGCGACTTCA, reverse 5′-GGTGGTCCAGG GTTTCTTA.

Grip Strength Test

The grip strength was measured using a grip strength meter (Yiyan Co. Ltd., YLS-13A). Mice were allowed to hold on to a metal grid and were gently pulled backwards by the tail at a constant speed until the mice could no longer hold the grid. Each mouse was given five trials, and the average value was used to represent the grip strength of an individual mouse.

Barnes Maze Assay

Male mice in the 12 M group were subjected to the Barnes maze assay to evaluate spatial memory function. For the Barnes maze assay, mice were trained to find a hole that connected to a black escape box, which was positioned around the circumference of a circular platform. The circular platform was 91 cm diameter and 0.4 cm thick, with 20 evenly distributed 5 cm diameter holes around the edge, with two overhead lights served as an aversive stimulus. Each trial was recorded by a video camera installed over the platform. Procedures were similar as described by Rosenfeld et al with modifications⁴⁴.

Albumin Purification

Serum samples of indicated groups were diluted with 20 mM Tris buffer containing 0.15 M NaCl at pH 7.8 before applying to a pre-equilibrated Blue BestaroseTMFF column (Bestchrom), followed by 3-bed volumes wash of nonspecific binding proteins. Mouse albumin was eluted by elution buffer (0.2 M NaSCN, pH 8.0), then dialyzed against PBS and concentrated by Amicon® ultra centrifugal filters with Ultracel-30 regenerated cellulose membrane (MerckMillipore, UFC803008) at 4° C. Protein concentrations were determined by the Pierce™ BCA Protein Assay Kit according to manufacturer's instructions (Thermo Scientific, 23227). Samples were analyzed on a Q-TOF mass spectrometer (Waters, SYNAPT G2-Si) instrument optimized for high-mass protein molecular weight analysis.

Immunofluorescence Assay

Frozen sections of mice which were dissected from mice, fixed with cold acetone. Then these samples were blocked with 10% goat serum and stained with primary antibodies overnight at 4° C. followed by the appropriate secondary fluorescently labeled antibodies at 4° C. overnight. Slides were stained with FITC-, Alexa 555- or Alexa 647-conjugated secondary antibodies, and nuclei were stained by DAPI. Fluorescence imaging was performed on Nikon A1 laser scanning confocal microscope and was analyzed with NIS-Elements Software (Nikon).

The following antibodies were used: mouse monoclonal antibody against phosphorylated tau (Thermo Scientific, MN1020), and anti-MHC1 antibody (Sigma-Aldrich, M 8421).

Aging-Related Parameters Determination

The Ellman's method was used to determine the content of free thiols⁴⁵. Mouse serum albumin and rMSA were mixed with equal volumes of 5, 5′-Dithiobis-(2-nitrobenzoic acid) (DTNB) reagent, respectively. The volume and concentration of DTNB used in this study were 100 μL and 2 mM, respectively. 800 μL Tris buffer (1 M) was added to make the volume of the reaction system reach 1000 μL. Samples were kept at room temperature for 30 min. The fluorescence absorbance was measured at 412 nm. Carbonyls in protein samples were quantified using the Protein Carbonyl Content Assay Kit (Abcam, ab126287) according to the manual. Homocysteine concentrations were measured by the enzyme-linked immunosorbent assay (ELISA) according to manufacturer's instructions (MEIMIAN, 1213). Concentrations of AGE were measured with an ELISA kit according to manufacturer's instructions (CLOUD-CLONE Co., CEB353Ge).

Statistical Analysis

The Kaplan-Meier method was used for survival analysis and the survival curves were compared by using the log-rank (Mantel-Cox) test. Statistically significant differences between groups were determined using a 2-tailed unpaired Student t-test or ANOVA, where p<0.05 is considered significant. Statistical analysis and diagramming were carried out by the Graphpad Prism 6.01 software unless otherwise noted.

### REFERENCES

1. Ludwig, F. C. & Elashoff, R. M. Mortality in syngeneic rat parabionts of different chronological age. Trans N Y Acad Sci. 34, 582-587 (1972).
2. Egerman, M. A. et al. GDF11 Increases with Age and Inhibits Skeletal Muscle Regeneration. Cell Metab. 22, 164-174 (2015).
3. Villeda, S. A. et al. Young blood reverses age-related impairments in cognitive function and synaptic plasticity in mice. Nat. Med. 20, 659-663 (2014).
4. Rattan, S. I. Increased molecular damage and heterogeneity as the basis of aging. Biol. Chem. 389, 267-272 (2008).
5. Yin, D. & Chen, K. The essential mechanisms of aging: Irreparable damage accumulation of biochemical side-reactions. Exp. Gerontol. 40, 455-465 (2005).
6. Lehallier, B. et al. Undulating changes in human plasma proteome profiles across the lifespan. Nat. Med. 25, 1843-1850 (2019).
7. Zimniak, P. What is the Proximal Cause of Aging? Front Genet. 3, 189 (2012).
8. Korovila, I. et al. Proteostasis, oxidative stress and aging. Redox Biol. 13, 550-567 (2017).
9. Reeg, S. & Grune, T. Protein Oxidation in Aging: Does It Play a Role in Aging Progression? Antioxid Redox Signal. 23, 239-255 (2015).
10. Hildebrandt, W., Kinscherf, R., Hauer, K., Holm, E. & Droge, W. Plasma cystine concentration and redox state in aging and physical exercise. Mech. Ageing Dev. 123, 1269-1281 (2002).
11. Garcia-Martinez, R. et al. Albumin: pathophysiologic basis of its role in the treatment of cirrhosis and its complications. Hepatology. 58, 1836-1846 (2013).
12. Hipkiss, A. R. Glycation, ageing and carnosine: are carnivorous diets beneficial? Mech. Ageing Dev. 126, 1034-1039 (2005).
13. Ahmed, N. Advanced glycation endproducts—role in pathology of diabetic complications. Diabetes Res Clin Pract. 67, 3-21 (2005).
14. Suji, G. & Sivakami, S. Glucose, glycation and aging. Biogerontology. 5, 365-373 (2004).
15. Uribarri, J. et al. Circulating glycotoxins and dietary advanced glycation endproducts: two links to inflammatory response, oxidative stress, and aging. J Gerontol A Biol Sci Med Sci. 62, 427-433 (2007).
16. Berlett, B. S. & Stadtman, E. R. Protein oxidation in aging, disease, and oxidative stress. J. Biol. Chem. 272, 20313-20316 (1997).
17. Chevion, M., Berenshtein, E. & Stadtman, E. R. Human studies related to protein oxidation: protein carbonyl content as a marker of damage. Free Radic Res. 33 Suppl, S99-S108 (2000).
18. Pandey, K. B., Mehdi, M. M., Maurya, P. K. & Rizvi, S. I. Plasma protein oxidation and its correlation with antioxidant potential during human aging. Dis. Markers. 29, 31-36 (2010).
19. McLean, R. R. et al. Homocysteine as a predictive factor for hip fracture in older persons. N Engl J Med. 350, 2042-2049 (2004).
20. Ostrakhovitch, E. A. & Tabibzadeh, S. Homocysteine and age-associated disorders. Ageing Res. Rev. 49, 144-164 (2019).
21. McCully, K. S. Vascular pathology of homocysteinemia: implications for the pathogenesis of arteriosclerosis. The American journal of pathology. 56, 111-128 (1969).
22. Seshadri, S. et al. Plasma Homocysteine as a Risk Factor for Dementia and Alzheimer's Disease. The New England Journal of Medicine. 346, 476-483 (2002).
23. Jakubowski, H. Homocysteine is a protein amino acid in humans. Implications for homocysteine-linked disease. J. Biol. Chem. 277, 30425-30428 (2002).
24. Jakubowski, H. Homocysteine Modification in Protein Structure/Function and Human Disease. Physiol. Rev. 99, 555-604 (2019).
25. Rothschild, M. A., Oratz, M. & Schreiber, S. S. Serum albumin. Hepatology. 8, 385-401 (1988).
26. Jr. Peters, T. All about albumin: Biochemistry, genetics, and medical applications; 1996.
27. Garcia Martinez, R. et al. Albumin: Pathophysiologic basis of its role in the treatment of cirrhosis and its complications. Hepatology. 58, 1836-1846 (2013).
28. Carballal, S. et al. Sulfenic Acid Formation in Human Serum Albumin by Hydrogen Peroxide and Peroxynitrite†. Biochemistry-US. 42, 9906-9914 (2003).
29. Turell, L., Radi, R. & Alvarez, B. The thiol pool in human plasma: The central contribution of albumin to redox processes. Free Radical Bio. Med. 65, 244-253 (2013).
30. Leto, S., Yiengst, M. J. & Barrows, J. C. H. The effect of age and protein deprivation on the sulfhydryl content of serum albumin. Journal of gerontology. 25, 4 (1970).
31. Era, S. et al. Age-related change in redox state of human serum albumin. Biochim Biophys Acta. 1247, 12-16 (1995).
32. Colombo, G. et al. Redox Albuminomics: Oxidized Albumin in Human Diseases. Antioxid. Redox Sign. 17, 1515-1527 (2012).
33. Jana, C. K., Das, N. & Sohal, R. S. Specificity of Age-Related Carbonylation of Plasma Proteins in the Mouse and Rat. Arch. Biochem. Biophys. 397, 433-439 (2002).
34. Wang, Z. et al. Age-related variations of protein carbonyls in human saliva and plasma: is saliva protein carbonyls an alternative biomarker of aging? Age. 37, (2015).
35. Byun, K. et al. Advanced glycation end-products produced systemically and by macrophages: A common contributor to inflammation and degenerative diseases. Pharmacol Ther. 177, 44-55 (2017).
36. Schalkwijk, C. G. & Stehouwer, C. Methylglyoxal, a Highly Reactive Dicarbonyl Compound, in Diabetes, Its Vascular Complications, and Other Age-Related Diseases. Physiol. Rev. 100, 407-461 (2020).
37. Glowacki, R. & Jakubowski, H. Cross-talk between Cys34 and lysine residues in human serum albumin revealed by N-homocysteinylation. J. Biol. Chem. 279, 10864-10871 (2004).
38. Sheng, J. et al. Novel Transgenic Mouse Model for Studying Human Serum Albumin as a Biomarker of Carcinogenic Exposure. Chem. Res. Toxicol. 29, 797-809 (2016).
39. Mehdipour, M. et al. Rejuvenation of three germ layers tissues by exchanging old blood plasma with saline-albumin. Aging (Albany N.Y.). 12, 8790-8819 (2020).
40. Abdulle, A. E. et al. Serum free thiols predict cardiovascular events and all-cause mortality in the general population: a prospective cohort study. BMC Med. 18, 130 (2020).
41. Damba, T. et al. Oxidative Stress Is Associated with Suspected Non-Alcoholic Fatty Liver Disease and All-Cause Mortality in the General Population. Liver Int., (2020).
42. Schalkwijk, C. G. & Stehouwer, C. Methylglyoxal, a Highly Reactive Dicarbonyl Compound, in Diabetes, Its Vascular Complications, and Other Age-Related Diseases. Physiol. Rev. 100, 407-461 (2020).
43. Yaffe, K. et al. Advanced glycation end product level, diabetes, and accelerated cognitive aging. Neurology. 77, 1351-1356 (2011).
44. Rosenfeld, C. S. & Ferguson, S. A. Barnes Maze Testing Strategies with Small and Large Rodent Models. Journal of Visualized Experiments, (2014).
45. Ellman, G. L. A colorimetric method for determining low concentrations of mercaptans. Arch. Biochem. Biophys. 74, 443-450 (1958).

1: A preparation of young and undamaged human serum albumin (HSA) that exhibits at least one or all of the following properties: (1) higher ratio of free thiol in Cys-34 residue, (2) lower level of advanced glycation end-product (AGE), (3) lower level of carbonylation, and (4) lower level of homocysteinylation, as compared to an endogenous HSA preparation obtained from a young individual of human.

2: The preparation of claim 1, wherein the young individual is below age of 30.

3: The preparation of claim 1, wherein the preparation exhibits at least one or all of the following properties: (1) the ratio of free thiol in Cys-34 residue being greater than 50%, as determined by the Ellman's method; (2) the level of advanced glycation end-product (AGE) being lower than 60 μg/g protein, as determined by ELISA (CLOUD-CLONE Co., CEB353Ge); (3) the level of carbonyl is lower than 1.7 nmol/mg protein, as determined by Protein Carbonyl Content Assay Kit (Abcam); and (4) the level of homocysteine is lower than 5 nmol/g protein as determined by ELISA (Jianglai, JL10022).

4: The preparation of claim 1, wherein the ratio of free thiol in Cys-34 residue is greater than 80%.

5: The preparation of claim 1, wherein the level of AGE is lower than 30 μg/g protein.

6: The preparation of claim 1, wherein the level of carbonyl is lower than 1.5 nmol/mg protein.

7: The preparation of claim 1, wherein the level of homocysteine is lower than 2 nmol/g protein.

8: The preparation of claim 1, wherein the ratio of free thiol in Cys-34 residue is greater than 80%, the level of AGE is lower than 30 μg/g protein, the level of carbonyl is lower than 1.5 nmol/mg protein, the level of homocysteine is lower than 2 nmol/g protein.

9: The preparation of claim 1, wherein the preparation is produced recombinantly or purified from plasma.

10-17. (canceled)

18: A method of extending lifespan and/or anti-aging in a subject, comprising the step of administering to the subject an effective amount of a preparation of young and undamaged human serum albumin (HSA), wherein the preparation exhibits at least one or all of the following properties: (1) higher ratio of free thiol in Cys-34 residue, (2) lower level of advanced glycation end-product (AGE), (3) lower level of carbonylation, and (4) lower level of homocysteinylation, as compared to an endogenous HSA preparation obtained from said subject.

19: The method of claim 18, wherein the preparation exhibits at least one or all of the following properties: (1) the ratio of free thiol in Cys-34 residue being greater than 50%, as determined by the Ellman's method; (2) the level of advanced glycation end-product (AGE) being lower than 60 μg/g protein, as determined by ELISA (CLOUD-CLONE Co., CEB353Ge); (3) the level of carbonyl is lower than 1.7 nmol/mg protein, as determined by Protein Carbonyl Content Assay Kit (Abeam); and (4) the level of homocysteine is lower than 5 nmol/g protein, as determined by ELISA (Jianglai, JL10022).

20: The method of claim 18, wherein the ratio of free thiol in Cys-34 residue is greater than 80%.

21: The method of claim 18, wherein the level of AGE is lower than 30 μg/g protein.

22: The method of claim 18, wherein the level of carbonyl is lower than 1.5 nmol/mg protein.

23: The method of claim 18, wherein the level of homocysteine is lower than 2 nmol/g protein, as determined by ELISA.

24: The method of claim 18, wherein the ratio of free thiol in Cys-34 residue is greater than 80%, the level of AGE is lower than 30 μg/g protein, the level of carbonyl is lower than 1.5 nmol/mg protein, the level of homocysteine is lower than 2 nmol/g protein.

25: The method of claim 18, wherein the subject is a patient or a healthy individual.

26: A pharmaceutical composition, comprising the preparation of young and undamaged human serum albumin as defined in claim 1 and a pharmaceutically acceptable carrier.

27: A method for monitoring the change of aging status of a subject, comprising:
obtaining a sample of human serum albumin (HSA) from the subject;
testing in the sample at least one or all of the following four parameters of HSA: (1) ratio of free thiol in Cys-34 residue, (2) level of advanced glycation end-product (AGE), (3) level of carbonylation, and (4) level of homocysteinylation;
if the ratio of free thiol in Cys-34 residue increases and/or the level of AGE, the level of carbonylation and/or the level of homocysteinylation decreases, as compared to the data from an earlier test from the same subject, it is determined that the aging status of the subject is improved.

28: A method of improving the function of skeletal muscle and/or improving the cognitive ability in a subject, comprising the step of administering to the subject an effective amount of a preparation of young and undamaged human serum albumin (HSA) as defined in claim 1.
